(19) United States
(12) Patent Application Publication
(10) Pub. No.: US 2007/0198031 A1
(43) Pub. Date: Aug. 23, 2007

(54) Method for performing dermabrasion

(76) Inventor: David Kellogg, Irvine, CA (US)

Correspondence Address: LAW OFFICES OF ERIC KARICH, 2807 ST. MARK DR., MANSFIELD, TX, 76063, US

(21) Appl. No.: 11/358,785

(22) Filed: Feb. 21, 2006

(51) Int. Cl. A61B 17/50

(52) U.S. Cl. 606/131

(57) ABSTRACT

A method of providing a dermabrasion to a person's skin includes the steps of first providing an ultrasonic dermabrasion device having a handle, a mounting surface positioned at a proximal end of the handle, a resilient pad positioned on the mounting surface, an ultrasonic vibrator, and a means for providing power to the ultrasonic vibrator. A fluid abrasion solution is applied to the person's skin, and the person's skin is rubbed with the resilient pad such that the ultrasonic vibrations are transmitted to the person's skin and, working in conjunction with the fluid abrasion solution, providing the dermabrasion to the person's skin.

### STATEMENT REGARDING FEDERALLY SPONSORED RESEARCH

Not Applicable

### CROSS-REFERENCE TO RELATED APPLICATIONS

Not Applicable

### BACKGROUND OF THE INVENTION

1. Field of the Invention

This invention relates generally to methods for performing a dermabrasion, and more particularly to a method for using an ultrasonic device for performing a dermabrasion.

2. Description of Related Art

Cleaning mechanisms that use ultrasonic vibrations to increase their cleaning effectiveness are known in the art, especially in the field of cleaning teeth. An example of such a cleaner is shown in Kleesattell, U.S. Pat. No. 4,281,987, which teaches an ultrasonically driven dental prophylaxis unit.

Ultrasonics are also used for various methods of treating skin. Examples of such uses are shown in the following:

Kellogg, U.S. Pat. No. 6,569,170, teaches a method of cleaning the skin using an ultrasonic cleaning device.

Liang et al., U.S. Pat. No. 5,012,797, teaches a method for removing wrinkles using an ultrasonic surgical tool that is adapted to abrade soft tissue.

Bock, WO 97/22325, teaches a method of using sonic and ultrasonic vibrations to increase the uptake of therapeutic agents through the skin.

Suroff, U.S. Pat. No. 4,040,414, teaches an ultrasonic personal care instrument with a suction device for sucking blockages out of pores to remove blackheads.

Other skin cleaning devices have included rotary motor cleaners that are well documented in the art. Examples of this design include Abura et al., U.S. Pat. No. 4,203,431 (facial treatment device), Wolff, U.S. Des. 245,948 (facial cleaning apparatus), Kawada, U.S. Pat. No. 3,906,940 (facial treatment device with oscillating rotary massaging member), Waters et al., U.S. Pat. No. 3,699,952 and Fry et a., U.S. Pat. No. 4,724,563 (skin treating appliance with orbitally driven brush).

Dermabrasions are typically provided using wire brushes, or fine sandpaper. Ultrasonic devices have not been used for providing dermabrasions.

The prior art teaches ultrasonic cleaners for cleaning teeth the skin and teeth; and the prior art teaches the use of wire brushes, diamond burrs, sandpaper, and similar cutting and scraping tools for providing dermabrasions. However, the prior art does not teach the use of an ultrasonic device for performing a dermabrasion. The present invention fulfills these needs and provides further related advantages as described in the following summary.

### SUMMARY OF THE INVENTION

The present invention teaches certain benefits in construction and use which give rise to the objectives described below.

The present invention provides a method of providing a dermabrasion to a person's skin. The method comprises the steps of first providing an ultrasonic dermabrasion device having a handle, a mounting surface positioned at a proximal end of the handle, a resilient pad positioned on the mounting surface, an ultrasonic vibrator positioned within a hollow interior and operably attached to the mounting surface for transmitting ultrasonic vibrations, and a means for providing power to the ultrasonic vibrator. A fluid abrasion solution is applied to the person's skin, and the person's skin is rubbed with the resilient pad such that the ultrasonic vibrations are transmitted to the person's skin and, working in conjunction with the fluid abrasion solution, providing the dermabrasion to the person's skin.

A primary objective of the present invention is to provide a method of providing a dermabrasion to a person's skin, the method having advantages not taught by the prior art.

Another objective is to provide a method of providing a dermabrasion to a person's skin using an ultrasonic dermabrasion device that utilizes ultrasonic vibrations to provide a more effective dermabrasion.

A further objective is to provide a fluid abrasive solution that operates in conjunction with the resilient pad to provide an effective dermabrasion.

Other features and advantages of the present invention will become apparent from the following more detailed description, taken in conjunction with the accompanying drawings, which illustrate, by way of example, the principles of the invention.

### BRIEF DESCRIPTION OF THE DRAWING

The accompanying drawings illustrate the present invention. In such drawings:

FIGURE 1 is a perspective view of the preferred embodiment of the present invention.

### DETAILED DESCRIPTION OF THE INVENTION

The above-described drawing figures illustrate the invention, a method of providing a dermabrasion of a person's skin 12 using an ultrasonic dermabrasion device 10 that transmits ultrasonic vibrations into the skin 12 through a resilient pad 32 that operate in conjunction with a fluid abrasive solution 38 to provide an effective dermabrasion to the person's skin 12.

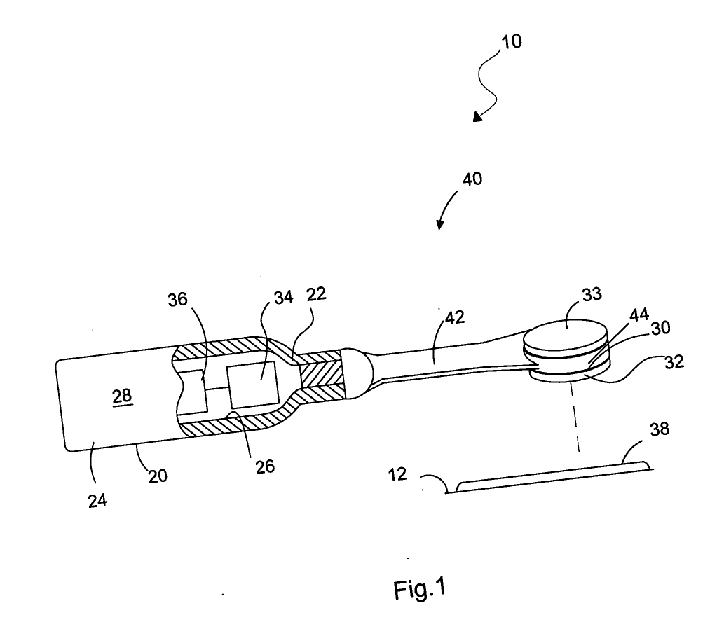

As shown in FIG. 1, the method utilizes an ultrasonic dermabrasion device 10 that includes a handle 20, a mounting surface 30, and a resilient pad 32. The handle 20 has a proximal end 22, a distal end 24, a hollow interior 26, and an exterior gripping surface 28. The handle is preferably formed of molded plastic, although other suitably strong and durable materials may also be used. In alternative embodiments, the handle 20 could also be a softer and more resilient ball shape, or a rigid metal housing, or any other equivalent material or shape that can be devised by those skilled in the art. A rubberized coating (not shown) could also be added to the exterior gripping surface 28 to facilitate gripping the ultrasonic dermabrasion device 10 if it gets wet during use, although this feature is not required.

The mounting surface 30 is positioned at the proximal end 22 of the handle 20, and the resilient pad 32 is positioned on the mounting surface 30. The mounting surface 30 is preferably in the form of a rigid paddle 40 that includes an elongate stem 42 that extends to a disk-shaped terminus 44. The elongate stem 42 is preferably relatively rigid and at least slightly resilient, for best transmitting the ultrasonic vibrations. The disk-shaped terminus 44 is shaped to receive the resilient pad 32, and may also include a second pad 33 mounted on the disk-shaped terminus 44 opposite the resilient pad 32. In the preferred embodiment, the resilient pad 32 has a texture and hardness that is different than the texture and hardness of the second pad, thereby enabling the user to select between two different textures and hardness—for example, a standard feel and one adapted for persons with sensitive skin.

An ultrasonic vibrator 34 is positioned within the hollow interior 26 and operably attached to the mounting surface 30 for transmitting ultrasonic vibrations. The ultrasonic vibrator 34 is preferably an eccentrically weighted motor, well known in the prior art, although any means for generating an ultrasonic vibration can be used. The ultrasonic vibrator 34 may be directly attached to the elongate stem 42, or it may be operably attached indirectly through the handle 20 and/or other components, as long as the ultrasonic vibrations are operably transmitted to the resilient pad 32.

For purposes of this application, the term ultrasonic shall be defined to include any form or vibrations or similar movement that is of sufficient speed and strength to perform the dermabrasion as described herein, including ultrasonic vibrations, sonic vibrations, and other forms and speeds of vibration that are operatively effective or equivalent.

The ultrasonic vibrator 34 is operably attached to a means for providing power 36 to the ultrasonic vibrator 34. The means for providing power 36 is preferably a rechargeable battery positioned within the hollow interior 26 and electronically connected to the ultrasonic vibrator 34. In an alternative embodiment, the means for providing power 36 may be a power cord (not shown) adapted to be connected to an external power outlet. Since the means for providing power is not a novel feature of the device, any practical method of powering the ultrasonic vibrator 34 should be considered within the scope of the present invention, and the matter is not described in any greater detail herein.

In use, the person's skin 12 is coated with the fluid abrasion solution 38. While we refer specifically to coating the person's skin 12 with the fluid abrasion solution 38, this terminology is expressly defined to include adding the fluid abrasion solution 38 to the resilient pad 32 and then using the ultrasonic dermabrasion device 10 to abrade the person's skin 12, or any other similar or equivalent methods.

Ultrasonic vibrations are then initiated in the ultrasonic dermabrasion device 10, the ultrasonic vibrations being transmitted from the ultrasonic vibrator 34, through the mounting surface 30, to the resilient pad 32. The person's skin 12 is then rubbed with the resilient pad 32 such that the ultrasonic vibrations are transmitted to the person's skin 12 and, working in conjunction with the fluid abrasion solution 38, providing the dermabrasion to the person's skin 12.

While the invention has been described with reference to at least one preferred embodiment, it is to be clearly understood by those skilled in the art that the invention is not limited thereto. Rather, the scope of the invention is to be interpreted only in conjunction with the appended claims.

What is claimed is:

1. A method of providing a dermabrasion of a person's skin, the method comprising the steps of:
a) providing an ultrasonic dermabrasion device comprising:
a handle having a proximal end, a distal end, a hollow interior, and an exterior gripping surface;
a mounting surface positioned at the proximal end of the handle;
a resilient pad positioned on the mounting surface;
an ultrasonic vibrator positioned within the hollow interior and operably attached to the mounting surface for transmitting ultrasonic vibrations; and
a means for providing power to the ultrasonic vibrator;
b) providing a fluid abrasion solution;
c) coating the person's skin with the fluid abrasion solution;
d) initiating the ultrasonic vibrations of the ultrasonic dermabrasion device, the ultrasonic vibrations being transmitted from the ultrasonic vibrator, through the mounting surface, to the resilient pad; and
e) rubbing the person's skin with the resilient pad such that the ultrasonic vibrations are transmitted to the person's skin and, working in conjunction with the fluid abrasion solution, providing the dermabrasion to the person's skin.

2. The method of claim 1 wherein the fluid abrasion solution includes abrasive crystals.

3. The method of claim 1 wherein the fluid abrasion solution includes aluminum oxide crystals.
